(19) United States
(12) Patent Application Publication
(10) Pub. No.: US 2024/0321422 A1
(43) Pub. Date: Sep. 26, 2024

(54) MASTER FORMULATION GENERATION FOR DRUG COMPOUNDING

(71) Applicant: Focal Pointe Data Solutions, LLC, Solana Beach, CA (US)

(72) Inventors: Kevin Scott PETTIT, Encinitas, CA (US); Mick Joseph HERMIZ, San Marcos, CA (US)

(21) Appl. No.: 18/611,036

(22) Filed: Mar. 20, 2024

Related U.S. Application Data
(60) Provisional application No. 63/453,404, filed on Mar. 20, 2023.

(51) Int. Cl. G16H 20/10; G16H 10/60

(52) U.S. Cl. CPC G16H 20/10; G16H 10/60

(57) ABSTRACT

Disclosed herein are system, method, and computer program product embodiments for generating missing master formulation records. An embodiment operates by receiving recipe data for a compounded preparation or drug product. The embodiment compares using a first artificial intelligence (AI) engine the recipe data with stored recipe data to determine whether a master formulation record corresponding to the recipe data is available. The embodiment generates, using a second AI engine, the master formulation record for the compounded preparation or drug product based on a determination that the master formulation record corresponding to the recipe data is not available. The embodiment receives an input comprising an approval or a correction for the generated master formulation record. The embodiment retrains the second AI engine based on the input. The embodiment finally generates, using the retrained second AI engine, a missing master formulation record for another recipe data.

### CROSS REFERENCE TO RELATED PATENT APPLICATION

This application claims the benefit of U.S. Provisional Patent Application No. 63/453,404, entitled “MASTER FORMULATION GENERATION FOR DRUG COMPOUNDING,” filed on Mar. 20, 2023, the entire contents of which are incorporated by reference herein in their entireties.

### BACKGROUND

According to the United States Pharmacopeia Chapters <795> and <797>, sterile and nonsterile compounding is defined as combining, admixing, diluting, pooling, reconstituting, repackaging, or otherwise altering a drug product or bulk drug substances to create a sterile and/or nonsterile pharmaceutical preparation. Also, according to other regulatory references drug compounding may also refer to altering the dosage form or delivery system of a drug, altering the strength of the drug, combining components or active ingredients and/or preparing a compounded drug preparation from chemicals or bulk drug substances.

A compounded preparation is provided to an individual patient in accordance with a written or an oral prescription order presented by an authorized prescriber. Products that are classified by the United States food and drug administration (FDA) to be demonstrably difficult to compound, products that appear on an FDA list of drugs that have been withdrawn or removed from the market because such drugs or components of drugs have been found to be unsafe or not effective, and drugs that are a copy or essentially a copy of one or more commercially available drug products (unless the drug is in short supply an on the FDA short supply list) shall not be compounded. Compounding provides patients with drug products that are customized to fit their unique clinical needs when commercially products are not available. For example, a patient may require a different strength or dosage form (e.g., a geriatric or a pediatric patient).

Sterile and nonsterile drug products can be compounded in a variety of places, including but not limited to hospitals and other healthcare institutions, medical and surgical patient treatment sites, infusion facilities, pharmacies, and physician, dental and veterinarian practice sites. Healthcare professionals that compound drug products in the aforementioned locations, include but are not limited to pharmacists, technicians, nurses, physicians, veterinarians, dentists, naturopaths, and chiropractors.

Compounding of drug products is regulated by federal, state laws and other regulatory agencies that provide laws, regulations, standards and guidelines for compounding sterile and nonsterile products. Many of the States, Boards of Pharmacy, and the United States Pharmacopeia (USP) require a Master Formulation Record (MFR) be created, reviewed, and approved prior to compounding a drug product. A MFR is a detailed record of procedures that describes how the sterile and nonsterile drug products are to be prepared. According to the guidelines provided by USP and enforced by the State Boards of Pharmacy and other regulatory bodies, an MFR must include at minimum the following information: name, strength or activity, and dosage form, identities and amounts of all active ingredients; if applicable, relevant characteristics of components (e.g., particle size, salt form, purity grade, solubility), inactive ingredients used, type and size of container closure system(s), complete instructions for preparing, including equipment, supplies, a description of the compounding steps, and any special precautions, physical description of the final product, Beyond Use Date (BUD), storage and handling requirements, reference source to support the stability, quality control (QC) procedures (e.g., pH testing, filter integrity testing), auxiliary labels to be applied, any required post-compounding processes or procedures, and other information as needed to describe the compounding process and ensure repeatability (e.g., adjusting pH and tonicity; sterilization method, such as steam, dry heat, irradiation, or filter).

Conventional approaches to create the MFR suffer from various technological problems. In most locations where drug compounding occurs, including pharmacies, compliance with having a MFR prior to a drug product being compounded has proven to be extremely difficult. This is a risky practice which has caused compounding errors that have led to untoward events that have resulted in patient harm, including death. Many practice sites, including hospitals, pharmacies, ambulatory care surgical facilities, infusion centers and physician office practices do not have the necessary resources and expertise to manage and maintain MFRs effectively and efficiently to ensure full compliance with having a MFR for every drug product prior to it being compounded. In some cases, where MFRs have been created expeditiously and were not thoroughly reviewed and approved have resulted in preventable errors caused by incomplete procedures/instructions, poor quality, and instability, which potentially could cause significant patient harm, including death.

### SUMMARY

Provided herein are system, apparatus, article of manufacture, method and/or computer program product embodiments, and/or combinations and sub-combinations thereof, for efficiently and accurately generating a master formulation record for a compounded preparation or drug product.

In some aspects, a method for master formulation record generation includes receiving recipe data for a compounded preparation or drug product, comparing, using a first artificial intelligence (AI) engine, the recipe data with stored recipe data to determine whether a master formulation record corresponding to the recipe data is available, generating, using a second AI engine, the master formulation record for the compounded preparation or drug product based on a determination that the master formulation record corresponding to the recipe data is not available, receiving an input comprising an approval or a correction for a generated master formulation record, retraining the second AI engine based on the input, and generating, using the retrained second AI engine, a missing master formulation record for another recipe data.

In some aspects, a system comprises a memory and at least one processor coupled to the memory. The at least one processor is configured to receive recipe data for a compounded preparation or drug product, compare, using a first AI engine, the recipe data with stored recipe data to determine whether a master formulation record corresponding to the recipe data is available, and generate, using a second AI engine, the master formulation record for the compounded preparation or drug product based on a determination that the master formulation record corresponding to the recipe data is not available. To train the second AI engine the at least one processor is configured to receive an input comprising an approval or a correction for a generated master formulation record corresponding to a missing master formulation record and retrain the second AI model based on the input.

Further features of the present disclosure, as well as the structure and operation of various embodiments, are described in detail below with reference to the accompanying drawings. It is noted that the present disclosure is not limited to the specific embodiments described herein. Such embodiments are presented herein for illustrative purposes only. Additional embodiments will be apparent to persons skilled in the relevant art(s) based on the teaching contained herein.

### BRIEF DESCRIPTION OF THE DRAWINGS

The accompanying drawings are incorporated herein and form a part of the specification, illustrate the present disclosure and, together with the description, further serve to explain the principles of the present disclosure and to enable a person skilled in the relevant art(s) to make and use embodiments described herein.

FIG. 1 is a block diagram of a platform that continuously improves an artificial intelligence engine's generation of master formulation records associated with compounded preparations or drug products, according to some embodiments.

FIG. 2 is a flow diagram that shows the operations of the platform for generating missing master formulation records for compounded preparations or drug products, according to an embodiment according to some embodiments.

FIG. 3 is a flowchart that illustrates a process for matching recipe data with stored master formulation records, according to some embodiments.

FIG. 4 is a diagram that illustrates different attributes of a master formulation record, according to some embodiments.

FIG. 5 is a flowchart that illustrates a process for generating a master formulation record for a recipe associated with a compounded preparation or drug product, according to some embodiments.

FIGS. 6A-6C illustrate exemplary graphical user interfaces for the platform, according to some embodiments.

FIG. 7 is an example computer system useful for implementing various embodiments.

The features of the present disclosure will become more apparent from the detailed description set forth below when taken in conjunction with the drawings, in which like reference characters identify corresponding elements throughout. In the drawings, like reference numbers generally indicate identical or similar elements. Additionally, generally, the leftmost digit(s) of a reference number identifies the drawing in which the reference number first appears.

### DETAILED DESCRIPTION

This specification discloses one or more embodiments that incorporate the features of the present disclosure. The disclosed embodiment(s) are provided as examples. The scope of the present disclosure is not limited to the disclosed embodiment(s). Claimed features are defined by the claims appended hereto.

The embodiment(s) described, and references in the specification to “one embodiment,” “an embodiment,” “an example embodiment,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to affect such feature, structure, or characteristic in connection with other embodiments whether or not explicitly described.

Spatially relative terms, such as “beneath,” “below,” “lower,” “above,” “on,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations), and the spatially relative descriptors used herein may likewise be interpreted accordingly.

The term “about,” “approximately,” or the like may be used herein to indicate a value of a quantity that may vary or be found to be within a range of values, based on a particular technology. Based on the particular technology, the terms may indicate a value of a given quantity that is within, for example, 1-20% of the value (e.g., +1%, +5%+10%, +15%, or +20% of the value).

Embodiments of the disclosure may be implemented in hardware, firmware, software, or any combination thereof. Embodiments of the disclosure may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read-only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc. In the context of computer storage media, the term “non-transitory” may be used herein to describe all forms of computer-readable media, with the sole exception being a transitory, propagating signal.

As described above, drug compounding may be restricted by regulations until a master formulation record has been prepared. Conventional approaches to generate the master formulation record suffer from various technological problems as described further below. Provided herein are system, apparatus, device, method, and/or computer program product embodiments, and/or combinations and sub-combinations thereof, for efficiently and accurately generating a master formulation record for a compounded preparation or drug product. Additionally, system, apparatus, device, method and/or computer program product embodiments, and/or combinations and sub-combinations thereof enable an artificial intelligence (AI) engine to continuously improve its generation of a missing master formulation record (also referred to herein as formulation) for a compounded preparation or drug product using feedback loops. It improves the technological field of drug compounding and the functioning of computing systems that are designed to maintain master formulation records and medical records as described further below.

As discussed above, conventional approaches to generate the master formulation record suffer from various technological problems. Master formulation records cannot be adequately prepared by a pharmacist for rarely compounded medications and/or for medications that require complicated compounding steps. The pharmacist does not have the required data that is needed to prepare the master formulation record. Thus, if the master formulation record for the rarely compounded medication is not previously available, the pharmacist may not be able to generate the master formulation record in order to compound the medication. In addition, matching recipe data to a master formulation record suffers from technological challenges. For example, each medical professional may write the recipe data differently (e.g., order of the ingredients, name of the ingredients).

The present disclosure provides an improvement in the technological field of drug compounding by providing an improved trained AI engine (also referred to as AI model) that solves the above-noted technological problems. The accuracy of the AI engine may be continuously improved by providing a feedback to the AI engine that improves the accuracy by developing a synonyms database and increasing the size of the training dataset using validated recipe-formulation sets. A validated recipe-formulation set may refer to a recipe and corresponding master formulation record that have been confirmed or verified by a medical expert (e.g., a pharmacist). Recipe or recipe data may refer to the medication (e.g., drug) included in an order.

The present disclosure also solves the technological problem of inconsistencies in the technological field of drug compounding by providing the feedback to the AI engine that continuously adds to the training data set of the AI engine. Further, to improve the AI engine's generation of the master formulation record, the feedback can utilize matched data recipes and master formulation records from a plurality of medical facilities (e.g., pharmacies, hospitals). This provides the advantage of generating improved master formulation records for rarely compounded medications, which may not be possible using conventional techniques.

In addition to the technological improvements and advantages described above, the present disclosure provides the technological advantage of generating master formulation records for compounded preparations or drug products in a timely fashion to comply with regulations without negatively affecting the patient. The quality of healthcare is improved as the generation of the master formulation record is available prior to compounding and is not tied only to the individual technician's knowledge and experience, thereby ensuring consistent quality and efficacy while reducing errors in dosage and formulation for each compounded preparation or drug product. Technicians can work more efficiently, as they do not need to spend time researching or formulating the compounded preparation or drug product. The availability of a master formulation record prior to compounding decreases the risk of errors related to incorrect ingredients or proportions, thereby enhancing patient safety. Furthermore, the approval of the generated master formulation record by a pharmacist prior to compounding adds an additional layer of quality control. Overall, having a master formulation record available prior to compounding streamlines the process, improves quality, and enhances patient safety.

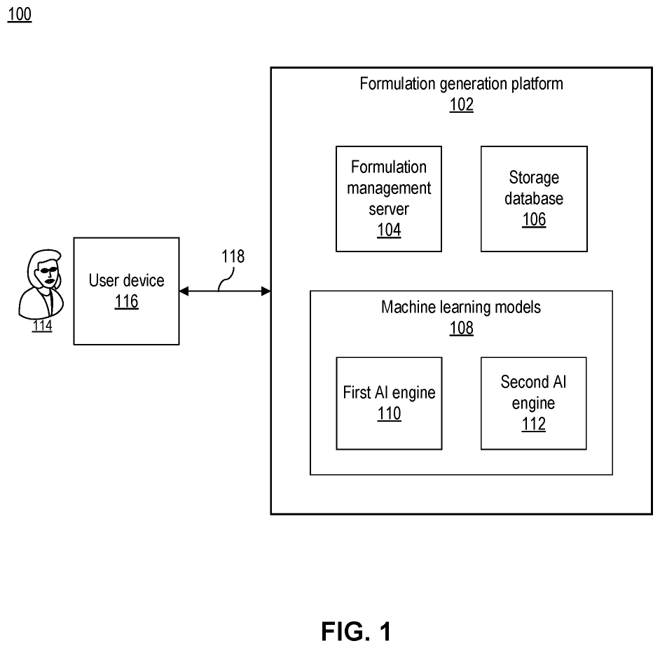

FIG. 1 is a block diagram of an environment 100 for generating a missing master formulation record in accordance with aspects of the present disclosure. Environment 100 includes a formulation generation platform 102. Platform 102 includes a formulation management server 104, a storage database 106, and machine learning models 108. Platform 102 may be a cloud-based platform (e.g., Amazon Web Services®) accessed via an application programming interface (API).

Machine learning models 108 may include a first AI engine 110 and a second AI engine 112. A user 114 may interact with platform 102 using a user device 116. For example, user 114 may interact with platform 102 via a mobile application installed on user device 116, a website that may be accessed from user device 116, or other communication interface.

User device 116 and platform 102 can be configured to communicate with each other via a network 118. User device 116 can include, but is not limited to, a desktop computer, laptop, smartphone, tablet, touchpad, wearable electronic device, smart watch, or other electronic device. It is to be appreciated that environment 100 may include other electronic devices in addition to or in place of the electronic devices illustrated in FIG. 1 without departing from the scope and spirit of this disclosure.

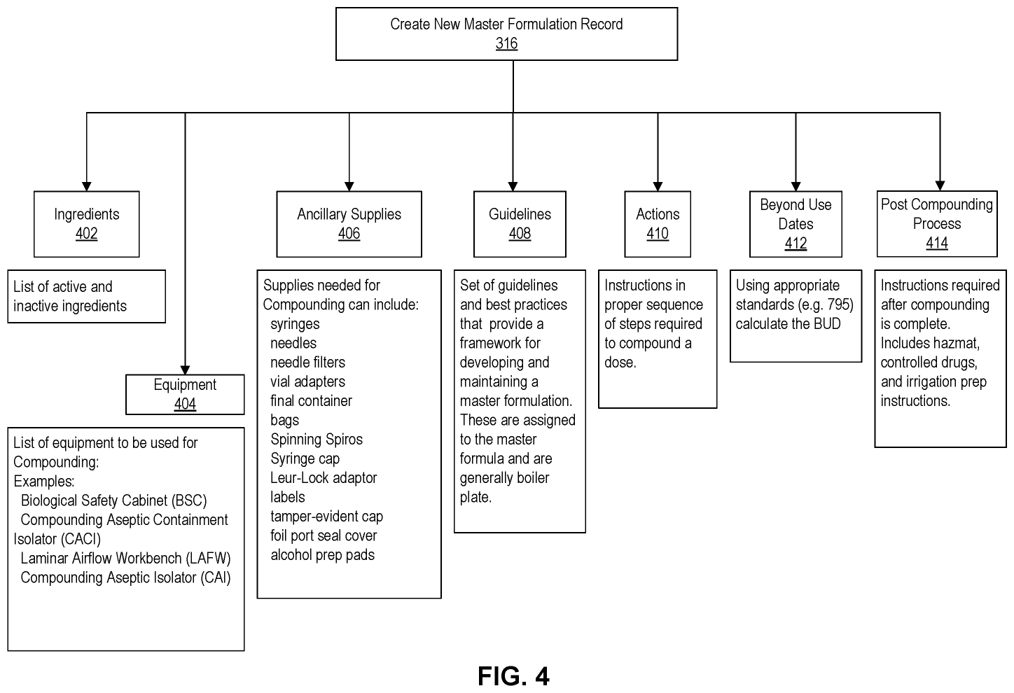

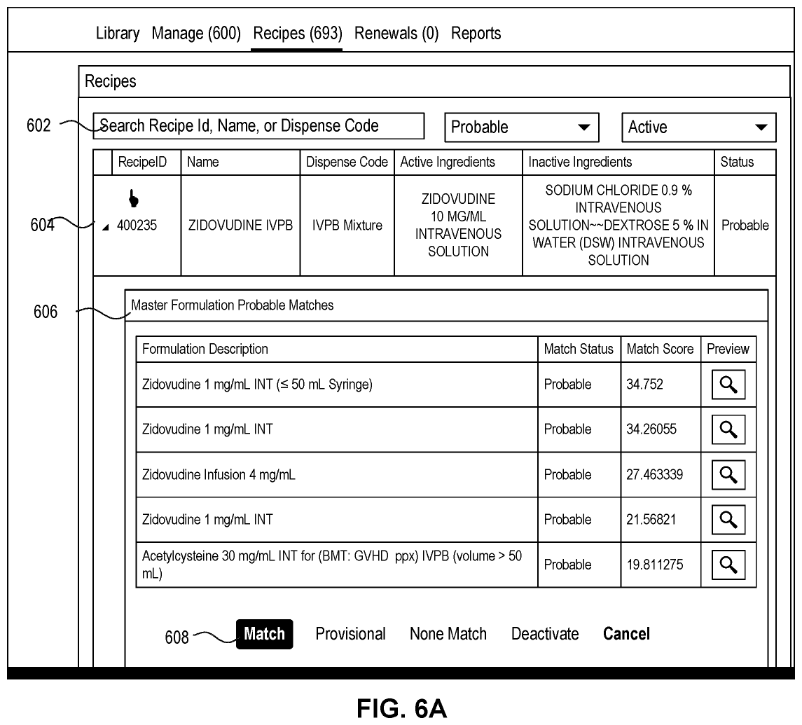

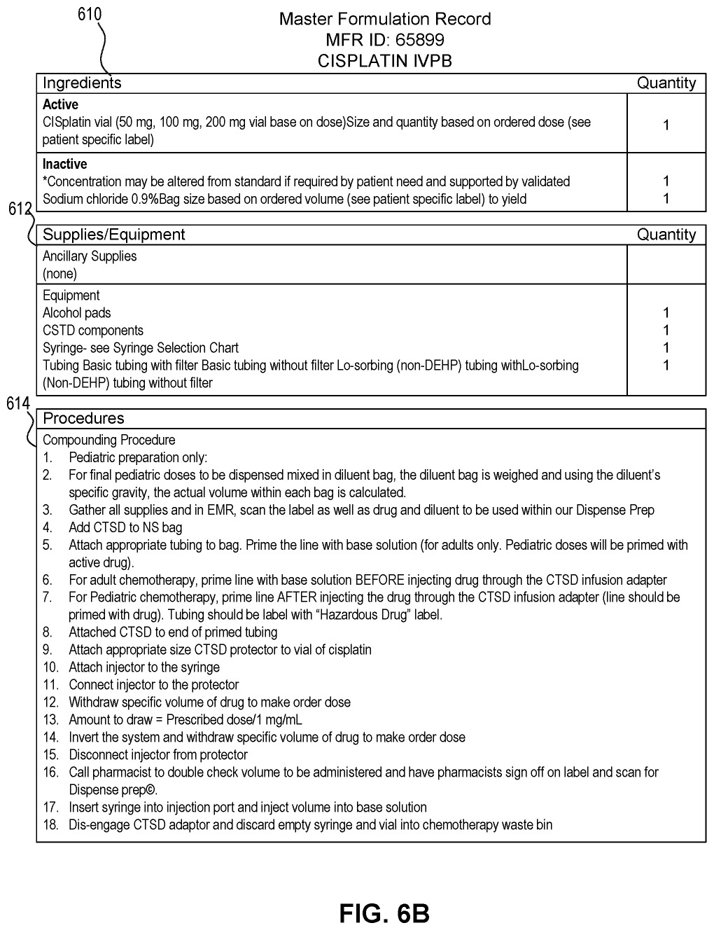

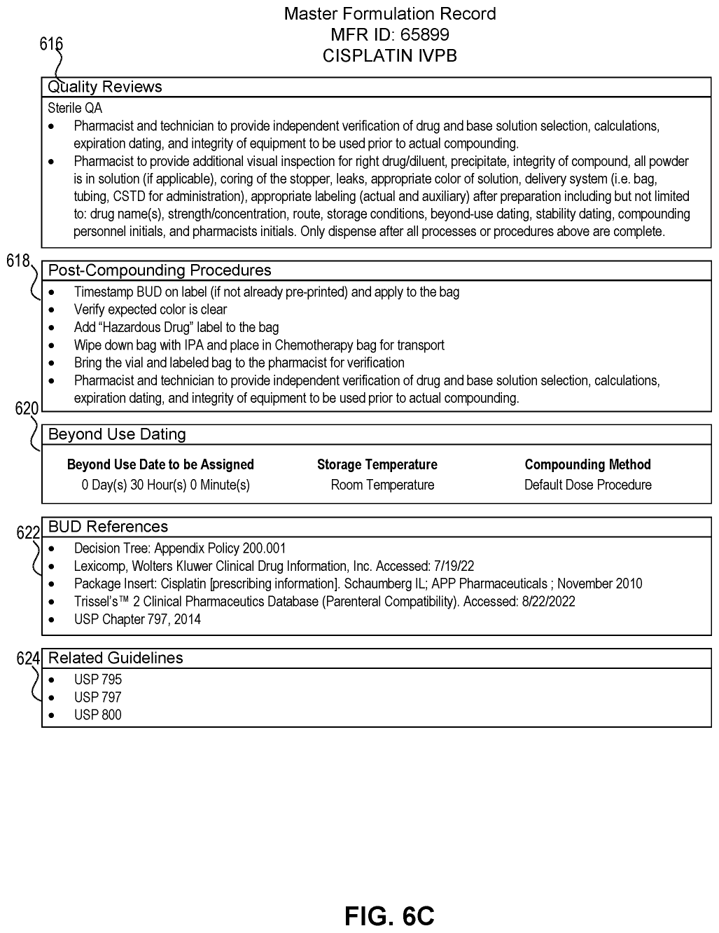

Platform 102 may receive an order or recipe from user 114 (e.g., medical professional, licensed physician) and may match or generate a master formulation record for the order. The order may refer to a prescription for a medication prepared by the medical professional and is associated with a patient. A recipe may refer to a basic description and ingredients of the medication. A master formulation, formulation, or master formulation record (MFR) may refer to a set of instructions for compounding the preparation or drug product. An exemplary master formulation record generated by platform 102 is shown in FIGS. 6B-6C. The master formulation record may comprise multiple parts or attributes (e.g., ingredients, instructions) as described further in relation to FIG. 4. The attributes are based on regulatory requirements.

Based on the received order, the master formulation record may be retrieved or generated using machine learning models 108. First AI engine 110 can determine whether there is a match between the received order and stored master formulation records in storage database 106. In response to determining that the master formulation record is missing, second AI engine 112 generates the master formulation record for the order. In some aspects, second AI engine 112 may represent one or more AI engines. For example, second AI engine 112 may include a plurality of AI engines and each AI engine of the plurality of engines may be configured to generate a part of the master formulation record. For example, a first engine may be configured to generate all the steps used in the compounding process and a second engine may be configured to generate a list of inactive ingredients. Once retrieved or generated the master formulation record may be output to user device 116. The master formulation record may be output on a display of user device 116. In some aspects, the master formulation record may be output to an external equipment or system for automated compounding of the medication.

First AI engine 110 and second AI engine 112 can be trained using data from one or more data sources using machine-learning techniques. As would be appreciated by a person of ordinary skill in the art, first AI engine 110 and second AI engine 112 can be trained using various machine learning techniques including, but not limited to, decision tree learning, association rule learning, artificial neural networks, deep learning, inductive logic programing, support vector machines, clustering, Bayesian network, reinforcement learning, representation learning, similarity, and metric learning, sparse dictionary learning, rule-based based machine learning, and learning classifier systems.

Data are continuously being added to the training set. Thus, an approval or rejection by the medical professional is added to the training set of machine learning models 108. For example, data are added to first AI engine 110 and/or second AI engine 112 and first AI engine 110 and/or second AI engine 112 are retrained.

First AI engine 110 and second AI engine 112 can be trained using supervised machine learning or unsupervised machine learning. In supervised machine learning, first AI engine 110 and second AI engine 112 can be trained using data that includes example inputs and desired outputs. For example, the example input can be recipe data, and the desired output can be a master formulation record approved by a pharmacist. In some aspects, first AI engine 110 and second AI engine 112 may be based on Apache Lucene.

In unsupervised machine learning, first AI engine 110 and second AI engine 112 can be trained using unlabeled data. Unlabeled data may not contain example inputs and desired outputs. Second AI engine 112 may attempt to determine one or more parts of the master formulation record.

In some aspects, platform 102 may receive a request from user 114 (e.g., pharmacist, auditor) to display the master formulation record associated with an order. Formulation management server 104 may retrieve the master formulation record and output the master formulation record on a display of user device 116. Formulation management server 104 may look up the formulation record using a recipe name or a unique identifier associated with the medication. In some aspects, user 114 may retrieve the master formulation record using a patient identifier. In addition to the master formulation record, other information including the pharmacist involved in the compounding, date, prescription number, label information may be documented by formulation management server 104.

In some aspects, platform 102 may provide a messaging feature (e.g., chat feature, email messaging, instant messaging) for health professionals to engage in. A pharmacist may put notes or questions for other pharmacists to collaborate on. The notes may be associated with a master formulation record. Another pharmacist may retrieve the notes associated with the master formulation record. In addition, another pharmacist can retrieve a contact of the pharmacist that approved the master formulation record. Then, both pharmacists can collaborate using the messaging feature. This provides the advantage of improving the quality of drug compounding specially for medication that are rarely compounded and for medication that require complicated compounding steps.

In some aspects, platform 102 may be implemented on a single user product with both client and server components residing on the same computer. That is, the operations of platform 102 may be performed by the user device 116. A user may download an application to the user device 116 and receive the generated and/or retrieved master formulation record.

As used herein, an application programming interface (“API”) may comprise any software capable of performing an interaction between one or more software components as well as interacting with and/or accessing one or more data storage elements (e.g., server systems, databases, hard drives, and the like). An API may comprise a library that specifies routines, data structures, object classes, variables, and the like. Thus, an API may be formulated in a variety of ways and based upon a variety of specifications or standards, including, for example, POSIX, the MICROSOFT WINDOWS API, a standard library such as C++, a JAVA API, and the like.

In some aspects, formulation management server 104 may represent one or more servers. For example, a server may be a mobile device, a laptop computer, a desktop computer, grid-computing resources, a virtualized computing resource, cloud computing resources, peer-to-peer distributed computing devices, a server farm, or a combination thereof. The servers may be centralized in a single room, distributed across different rooms, distributed across different geographic locations, or embedded within the network 118. The servers can couple with the network 118 to communicate with other devices, such as a client device or user device 116. The servers and the client device may be stand-alone devices and work independently from one another.

Network 118 refers to a telecommunications network, such as a wired or wireless network. Network 118 can span and represent a variety of networks and network topologies. For example, network 118 can include wireless communication, wired communication, optical communication, ultrasonic communication, or a combination thereof. For example, satellite communication, cellular communication, Bluetooth, Infrared Data Association standard (IrDA), wireless fidelity (WiFi), and worldwide interoperability for microwave access (WiMAX) are examples of wireless communication that may be included in network 118. Cable, Ethernet, digital subscriber line (DSL), fiber optic lines, fiber to the home (FTTH), and plain old telephone service (POTS) are examples of wired communication that may be included in network 118. Further, network 118 can traverse a number of topologies and distances. For example, network 118 can include a direct connection, personal area network (PAN), local area network (LAN), metropolitan area network (MAN), wide area network (WAN), or a combination thereof.

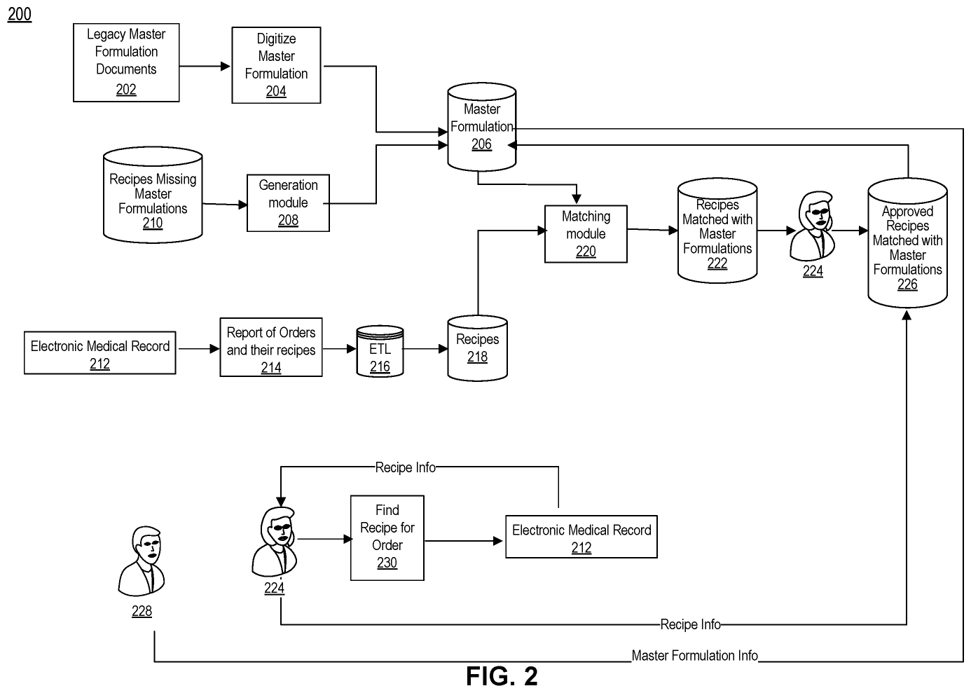

FIG. 2 is a flow diagram 200 that shows the operations of platform 102, according to an embodiment. Flow 200 may begin by receiving formulation documents 202. Formulation documents 202 may represent legacy master formulation documents or records. That is, master formulation documents that have been created and approved by medical professionals for a plurality of recipes. In some aspects, formulation documents 202 may be in printed form or scanned copies of documents. Formulation documents may be in an electronic format. In some aspects, the formulation documents may be Microsoft word documents, Microsoft word documents, or other documents type as would be understood by one of ordinary skill in the art. Digitization module 204 digitizes formulation documents 202. Digitization module 204 may parse formulation documents 202 to normalize data in a structured format. The digitized formulations are stored in a master formulations database 206. Master formulations database 206 stores the digitized formulations and master formulation records generated by platform 102.

An electronic medical record (EMR) 212 may output a report of orders and corresponding recipes 214 to a database 216. EMR 212 may refer to a digital record-keeping used by healthcare providers in hospitals to document and track patient medical information. In some aspects, the report of orders does not include patient information. Thus, platform 102 preserves the privacy of patients. In some aspects, database 216 may be an extract, transform, and load (ETL) database that combines data from multiple files (e.g., report of orders and associated recipes 214). Data from database 216 are loaded into a recipes database 218. Data in recipe database 218 are normalized. That is, the data are structured and may be used by a matching module 220. In some aspects, matching module 220 may correspond to first AI engine 110. Matching module 220 may receive as inputs recipe data stored in recipes database 218 and data stored in master formulations database 206. Matching module 220 determines whether there is a match between a master formulation record stored in master formulations database 206 and recipes database 218 (i.e., a recipe received from electronic medical record 212). In response to determining by the matching module 220 that there is a match, the matched recipe and master formulation record are stored in a database 222. Database 222 may represent a queue of matched master formulation records that are to be validated by a medical professional 224 (e.g., a pharmacist). Medical professional 224 may be the individual responsible for maintaining compliance with regulations. In response to determining that there is no match between the recipe and the stored master formulation record, data associated with the recipe are output to a recipes missing master formulation database 210.

A generation module 208 may take recipe data from the recipes missing master formulation database 210 and generate the corresponding master formulation record. After generating the missing master formulation record, the master formulation record is added to master formulations 206 and/or recipes matched with master formulation 222. In some aspects, generation module 208 may correspond to second AI engine 112.

Medical professional 224 may review recipes and master formulation record in database 222. Once approved the recipes are stored in database 226. The set of approved recipe and master formulation record is added to the training set of matching module 220. In addition, if the master formulation record is generated by generation module 208 and is approved by medical professional 224, the set is also added to the training set of the generation module 208.

Not only platform 102 is used for matching and generating missing master formulation record, platform 102 may be used to maintain data and records that may be retrieved during compliance checks and audits. An auditor 228 that enforces state regulations may ask medical professional 224 to retrieve the master formulation record used to prepare the medication of an order. A module 230 may retrieve the recipe data from EMR 212 using an identifier associated with the order. The identifier may be a unique alphanumeric string associated with the order. The recipe data may be retrieved and displayed on a user device associated with medical health professional 224. The recipe data may be used to retrieve the master formulation record from database 226. The retrieved master formulation record is output to auditor 228 and/or health professional 224.

In some aspects, databases 206, 210, 222, and 226 may correspond to storage database 106 of FIG. 1.

The modules described in FIG. 2 may be implemented as instructions stored on a non-transitory computer readable medium to be executed by one or more computing units such as a processor, a special purpose computer, an integrated circuit, integrated circuit cores, or a combination thereof. The non-transitory computer readable medium may be implemented with any number of memory units, such as a volatile memory, a nonvolatile memory, an internal memory, an external memory, or a combination thereof. The non-transitory computer readable medium may be integrated as a part of formulation management server 104 or installed as a removable portion of formulation management server 104.

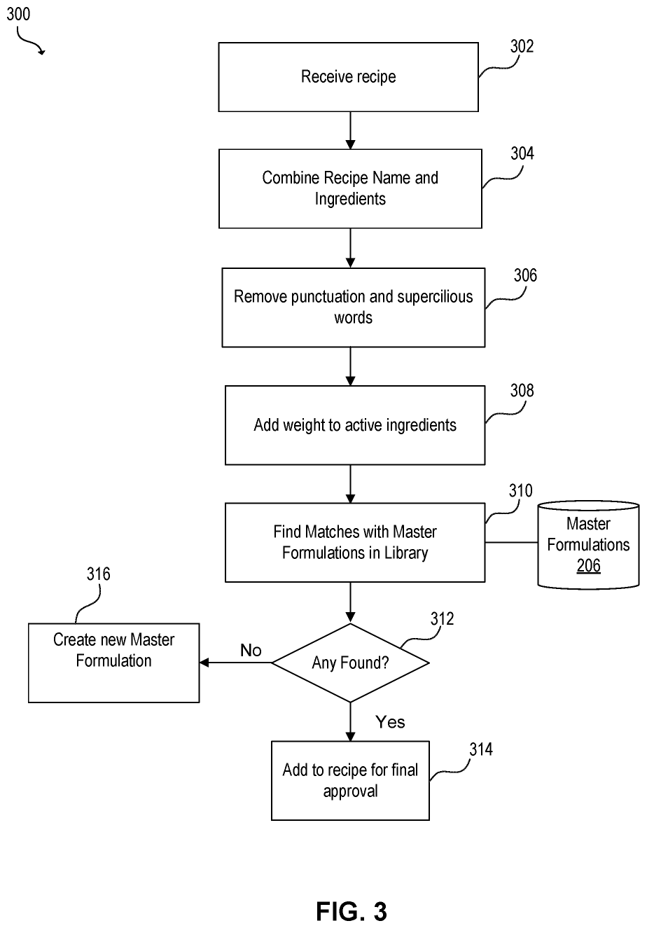

FIG. 3 is a flowchart for a method 300 for matching recipe data with stored master formulation records, according to an embodiment. Method 300 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 3, as will be understood by a person of ordinary skill in the art.

Method 300 shall be described with reference to FIGS. 1 and 2. However, method 300 is not limited to that example embodiment.

In 302, platform 102 may receive recipe data from an electronic device (e.g., electronic medical record 212 or user device 116. The received data is normalized. For example, formulation management server 104 may parse the text data and remove rich text before further processing. Matching module 220 may retrieve attributes that are used to match the recipe data with a master formulation record. For example, matching module 220 may retrieve a name of the recipe, a dispense code that indicates the form of the recipe, one or more inactive ingredients, one or more active ingredients, or the like.

In 304, matching module 220 may combine the recipe name and ingredients.

In 306, matching module 220 may remove punctuation and supercilious words from the combined recipe name and ingredients. Matching module 220 may determine that an ingredient is listed twice and is removed before proceeding to the matching step. In addition, text data that is not an input to the AI engine may be removed.

In 308, matching module 220 may add weight to active ingredients. The weights may be determined based on the training of the AI engine. For example, matching module may assign a higher weight to the active ingredients compared to the inactive ingredients. In some aspects, the weights assigned to the active ingredients may be double the weight assigned to the inactive ingredients. In addition to adding weights to the active ingredients, matching module 220 may remove some input (i.e., assign a zero weight) to other attributes based on the feedback. For example, some equipment may be removed.

Matching module 220 may be trained to recognize synonyms to active ingredients. Synonyms may include equivalent ingredients such as generic and brand name of an active ingredient. For example, matching module 220 may change “Motrin” or “Advil” in the active ingredient to “Ibuprofen” before comparing with stored recipe data. The synonyms may be stored in a synonym database. In some aspects, matching module 220 may identify the active ingredients by comparing the received data to a list of possible active ingredients. The list of possible active ingredients may be stored in database 106.

In 310, matching module 220 may compare the combined recipe name and ingredients with stored master formulation records in master formulation database 206. Matching module 220 may use a string matching technique to match the recipe name and ingredients of the stored master formulation records. For example, if the active ingredient includes Ibuprofen, the matching module 220 may retrieve one or more master formulation record where Ibuprofen is included in the active ingredient attribute.

In 312, a determination is made by matching module 220 to whether a formulation corresponding to the combined recipe name and ingredients is available in master formulation database 206. In some aspects, matching module 220 may determine a score for each stored master formulation record. The score indicates the probability that a master formulation record corresponds to the recipe data. In some aspects, matching module 220 may output to medical professional 224 a top k list of master formulation records. For example, matching module 220 may output the top five master formulation records with the highest probability score. In some aspects, matching module 220 may output all the master formulation records that have a probability score above a threshold. Formulation management server 104 may receive a selection from medical professional 224 that indicates the correct match. An exemplary interface showing the top k list is shown in FIG. 6A. The correct match and the recipe data are added to the training set via the feedback loop. Incorrect matches may also be added to the training set.

In some aspects, matching module 220 may identify the master formulation record for a recipe that is weight specific. For example, the master formulation record may be different for a recipe associated with a child versus an adult (e.g., weight based).

In some aspects, formulation management server 104 may receive an indication that two or more of the displayed master formulation records are the same. Matching module 220 may analyze the records in order to determine equivalent ingredients or equipment. In some aspects, the duplicate record is deleted from master formulation database 206.

In 314, in response to determining that a master formulation record corresponding to the combined recipe name and ingredients is available in the master formulation database 206, the recipe and corresponding record may be added to recipes matched with master formulations database 222.

In 316, in response to determining that the formulation corresponding to the combined recipe name and ingredients is not available in the master formulation database 206, the recipe data may be added to recipes missing master formulations database 210. Generation module 208 may create a master formulation record for each recipe data in the missing master formulation database 210. The attributes or parts of the master formulation record are generated as described in relation to FIG. 4.

FIG. 4 is a diagram that illustrates different attributes of the formulation, according to some aspects. The master formulation record can include a plurality of attributes or parts. As discussed above, platform 102 generates the master formulation record for the recipe data.

The attributes includes ingredients 402, equipment 404, ancillary supplies 406, guidelines 408, actions 410, beyond used dates (BUD) 412, and post compounding process 414. Some attributes may be generated by AI engines (e.g., second AI engine 112) using machine learning techniques, string matching techniques, or other techniques.

Ingredients 402 represents a list of active and inactive ingredients. Equipment 404 represents a list of equipment to be used for compounding. For example, the equipment 404 may include a biological safety cabinet (BSC), compounding aseptic containment isolator (CACI), a laminar airflow workbench (LAFW), and compounding aseptic isolator (CAI).
Second AI engine 112 identifies one or more equipment that is used for compounding the medication.

As mentioned above, attributes may also include ancillary supplies 406. Ancillary supplies 406 include supplies used during the compounding of the medication. For example, ancillary supplies 406 include a syringe, a needle, a needle filter, a vial adapter, a final container, a bag, a spinning spiro, a syringe cap, a luer-lock adapter, a label, a tamper-evident cap, a foil port seal cover, and an alcohol prep pad. The list of all supplies may be stored in storage database 106. Formulation management server 104 may identify the list of supplies based on the dispense code and the active ingredients.

Guidelines 408 includes a set of guidelines and best practices that provide a framework for developing and maintaining a master formulation record. The guidelines may include quality review steps performed at each step during compounding of the medication. For example, an initial quality review may include verifying all calculations and that ingredients and supplies are free of particle and discoloration. Some of the guidelines are common to all master formulation records. Some of the guidelines may be based on the dispense code. Thus, the guidelines may be retrieved based on the dispense code identified in the recipe data. Then, guidelines 408 of the master formulation record populated using the retrieved guidelines.

Actions 410 includes instructions in a proper sequence of steps required to compound a dose. The actions 410 may include a set of predefined steps (e.g., wait, draw, remove, connect, withdraw, transfer, clamp, open, inspect, dispose). Second engine 112 generates the instructions to compound the medication in the proper order. Second AI engine 112 may generate a complete instruction by associating a predefined step with an appropriate ingredient and a supply from ancillary supplies 306. For example, second AI engine 112 may generate “Draw up drug A with 60 ml syringe and 18 G needle.”

BUD 412 indicates the BUD calculated using an appropriate standard as would be understood by one of ordinary skill in the art. Formulation management server may identify the BUD of the compounded as equal to the shortest BUD of all the ingredients used in the compounded medication. The BUD of active ingredients and inactive ingredients may be stored in database 106.

Post compounding process 414 are instructions that may be performed after compounding is complete. The instructions includes hazmat, controlled drugs, and irrigation preparation instructions. The instructions may be identified based on the ingredients. Formulation management server 104 may store an association between an ingredient and a post compounding instruction. Formulation management server 104 may use a look-up table to retrieve the post compounding instruction for all the ingredients identified in the recipe data.

In some aspects, second AI engine 112 or formulation management server 104 may determine a storage method when the storage method for the medication is not included in the recipe data or associated order. In some aspects, the storage method may be determined based on the active and inactive ingredients of the medication. In some aspects, the storage method may correspond to the most restrictive method between the active and inactive ingredients. For example, if the storage method of an active ingredient is “refrigeration” while other ingredients do not have a specific storage method, then the storage method of the medication is identified as refrigeration. Further, the refrigeration temperature of the medication may be identified as the lowest temperature associated with an ingredient of the medication. The storage method and associated temperature (if any) may be stored in database 106 for a plurality of active and inactive ingredients. The formulation management server 104 may retrieve the storage method for all ingredients identified in the recipe data.

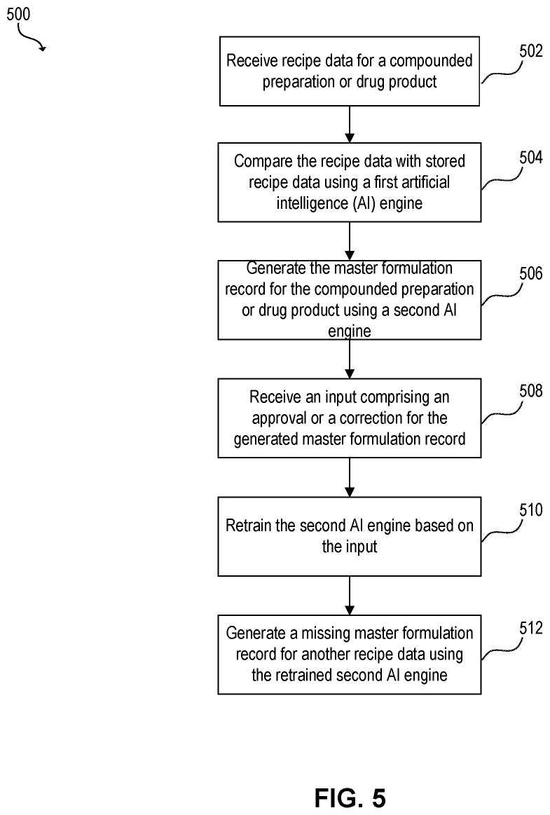

FIG. 5 is a flowchart for a method 500 for generating master formulation record, according to some aspects. Method 500 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 5, as will be understood by a person of ordinary skill in the art. In 502, platform 102 may receive recipe data for a compounded preparation or drug product.

In 504, first AI engine 110 may compare the recipe data with stored recipe data to determine whether a master formulation record corresponding to the recipe data is available. In some aspects, the recipe data are pursed to extract an active ingredient. A higher weight is assigned to a feature corresponding to the active ingredient compared to other features in first AI engine 110. In some aspects, first AI engine 110 may automatically correct one or more typographically errors in the recipe data before attempting to match the recipe data with a stored master formulation record.

In 506, second AI engine 112 may generate the master formulation record for the compounded preparation or drug product based on a determination that the master formulation record corresponding to the recipe data is not available.

In 508, platform 102 may receive an input from a user device associated with a health professional. The input comprises an approval or a correction for the generated master formulation record.

In 510, second AI engine 112 may be retrained based on the input received in 508.

In 512, second AI engine 112 may generate a missing master formulation record for another recipe data. As described previously herein, generating the master formulation record includes predicting one or more attributes of the master formulation record. The one or more attributes may include an ingredient list, an expiration date, an equipment, a set of guidelines, and instructions to compound the preparation or drug product.

FIGS. 6A-6C illustrate exemplary user interfaces for platform 102, in accordance with some aspects of the present disclosure. Graphical user interfaces shown in FIGS. 6A-6B are part of platform 102 and allow the user to display generated and retrieved master formulation records.

A user may be provided with a search box 602. The user may type an identifier of a recipe, a name of a recipe, or a dispense code. A first pane 604 may show the user retrieved recipe data that correspond to the entered data in search box 602. A second pane 606 may show the top-k master formulation records that have been identified by platform 102. The user may be presented with a control 608 to accept or reject each record of the master formulation records. Pane 606 may also show the match score for each master formulation record. The master formulation records may be sorted by descending order based on the match score.

FIGS. 6B-6C show an exemplary master formulation record generated by platform 102. A first pane 610 shows the list of active and inactive ingredients and the corresponding quantity. A second pane 612 shows the supplies and equipment and the quantity needed for compounding. A third pane 614 shows the procedure instructions. The instructions are shown in the order to be performed by the technician or pharmacist when compounding. A fourth pane 616 shows instructions for quality review. A fifth pane 618 shows the post-compounding procedure. A sixth pane 620 show the BUD identified or generated by platform 102. The BUD may also include a storage temperature and a compounding method. A seventh pane 622 shows a list of reference used in the BUD determination. An eight pane 624 shows a list of compounding guidelines that are associated with the recipe data.

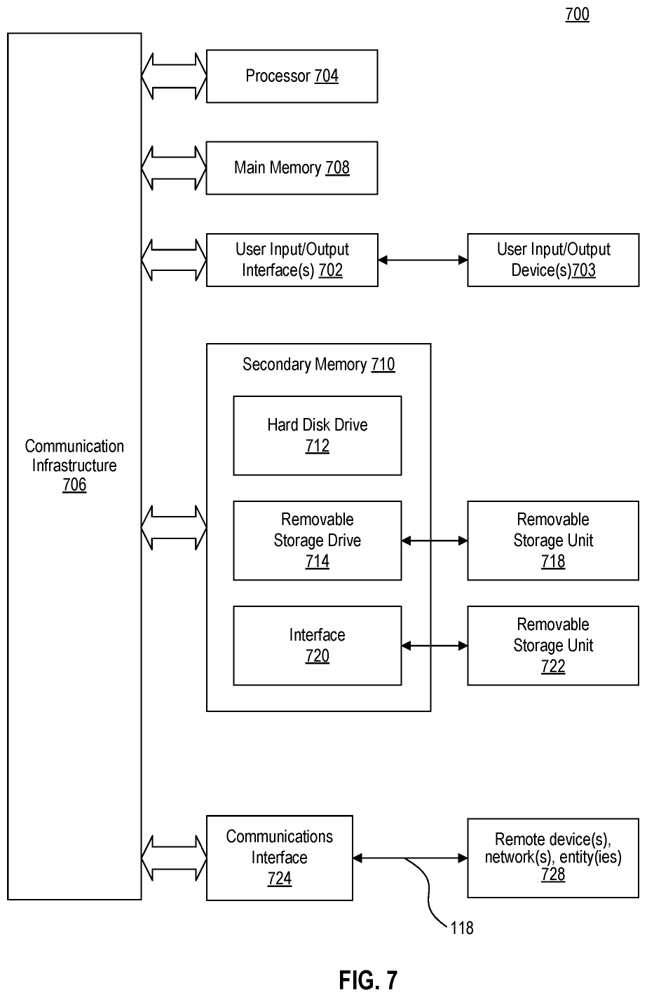

Various embodiments can be implemented, for example, using one or more computer systems, such as computer system 700 shown in FIG. 7. Computer system 700 can be used, for example, to implement method 300 of FIG. 3 and/or method 500 of FIG. 5. Computer system 700 can be any computer capable of performing the functions described herein.

Computer system 700 can be any well-known computer capable of performing the functions described herein.

Computer system 700 includes one or more processors (also called central processing units, or CPUs), such as a processor 704. Processor 704 is connected to a communication infrastructure or bus 706.

One or more processors 704 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 also includes user input/output device(s) 703, such as monitors, keyboards, pointing devices, etc., that communicate with communication infrastructure 706 through user input/output interface(s) 702.

Computer system 700 also includes a main or primary memory 708, such as random-access memory (RAM). Main memory 708 may include one or more levels of cache. Main memory 708 has stored therein control logic (i.e., computer software) and/or data. Computer system 700 may also include one or more secondary storage devices or memory 710. Secondary memory 710 may include, for example, a hard disk drive 712 and/or a removable storage device or drive 714. Removable storage drive 714 may be a floppy disk drive, a magnetic tape drive, a compact disk drive, an optical storage device, tape backup device, and/or any other storage device/drive.

Removable storage drive 714 may interact with a removable storage unit 718. Removable storage unit 718 includes a computer usable or readable storage device having stored thereon computer software (control logic) and/or data. Removable storage unit 718 may be a floppy disk, magnetic tape, compact disk, DVD, optical storage disk, and/any other computer data storage device. Removable storage drive 714 reads from and/or writes to removable storage unit 718 in a well-known manner.

According to an exemplary embodiment, secondary memory 710 may include other means, instrumentalities or other approaches for allowing computer programs and/or other instructions and/or data to be accessed by computer system 700. Such means, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 enables computer system 700 to communicate and interact with any combination of remote devices, remote networks, remote entities, etc. (individually and collectively referenced by reference number 728). For example, communication interface 724 may allow computer system 700 to communicate with remote devices 728 over communications path or network 118, which may be wired and/or wireless, and which may include any combination of LAN, WANs, the Internet, etc. Control logic and/or data may be transmitted to and from computer system 700 via network 118.

In an embodiment, a tangible, non-transitory apparatus or article of manufacture comprising a tangible, non-transitory computer useable or readable medium having control logic (software) stored thereon is also referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown

in FIG. 7. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

The breadth and scope of this disclosure should not be limited by any of the above-described exemplary embodiments, but should be defined only in accordance with the following claims and their equivalents.

What is claimed is:

1. A computer implemented method for formulation generation, comprising:
receiving, by at least one processor, recipe data for a compounded preparation or drug product;
comparing, using a first artificial intelligence (AI) engine, the recipe data with stored recipe data to determine whether a master formulation record corresponding to the recipe data is available;
generating, using a second AI engine, the master formulation record for the compounded preparation or drug product based on a determination that the master formulation record corresponding to the recipe data is not available;
receiving an input comprising an approval or a correction for the generated master formulation record;
retraining the second AI engine based on the input; and
generating, using the retrained second AI engine, a missing master formulation record for another recipe data.

2. The computer implemented method of claim 1, further comprising:
determining a match score between the recipe data and the stored recipe data;
displaying a list of one or more recipe for a user to select on a user device, wherein the displayed one or more recipe have a respective match score above a threshold;
receiving a selection from the user, wherein the selection indicates a correct match between the recipe data and the stored recipe data; and
retraining the first AI engine based on the selection of the user.

3. The computer implemented method of claim 2, further comprising:
determining that a first ingredient included in the recipe data and a second ingredient included in the stored recipe data are exchangeable based on the selection received from the user;
adding the first ingredient and the second ingredient to a synonyms database; and
retraining the first AI engine using the synonyms database.

4. The computer implemented method of claim 1, wherein the comparing further comprising:
parsing the recipe data to extract an active ingredient; and
assigning a higher weight to a feature corresponding to the active ingredient compared to weights corresponding to other features of the first AI engine.

5. The computer implemented method of claim 1, wherein the generating the master formulation record further comprising:
predicting one or more attributes of the master formulation record, wherein the attributes comprise at least one of an ingredient list, an expiration date, an equipment, a set of guidelines, and instructions to compound the preparation or drug product.

6. The computer implemented method of claim 1, further comprising:
automatically correcting one or more typographical errors in the recipe data before the comparing step.

7. The computer implemented method of claim 1, further comprising: receiving, from a user device, data that identifies a patient; retrieving the recipe data associated with the patient;
and
displaying one or more master formulation records associated with the patient.

8. The computer implemented method of claim 1, wherein the recipe data is received from an electronic medical record.

9. A system,
comprising: a
memory; and
at least one processor coupled to the memory and configured to:
receive recipe data for a compounded preparation or drug product;
compare, using a first artificial intelligence (AI) engine, the recipe data with stored recipe data to determine whether a master formulation record corresponding to the recipe data is available; and
generate, using a second AI engine, the master formulation record for the compounded preparation or drug product based on a determination that the master formulation record corresponding to the recipe data is not available, wherein to train the second AI engine the at least one processor is configured to:
receive an input comprising an approval or a correction for a generated master formulation record corresponding to a missing master formulation record; and
retrain the second AI engine based on the input.

10. The system of claim 9, wherein the at least one processor is further configured to: determine a match score between the recipe data and the stored recipe data;
display a list of one or more recipe for a user to select on a user device, wherein the displayed one or more recipe have a respective match score above a threshold;
receive a selection from the user, wherein the selection indicates a correct match between the recipe data and the stored recipe data; and
retrain the first AI engine based on the selection of the user.

11. The system of claim 10, wherein the at least one processor is further configured to: determine that a first ingredient included in the recipe data and a second ingredient
included in the stored recipe data are exchangeable based on the selection received from the user; add the first ingredient and the second ingredient to a synonyms database; and
retrain the first AI engine using the synonyms database.

12. The system of claim 9, wherein to compare the recipe data with the stored recipe data, the at least one processor is further configured to:
parse the recipe data to extract an active ingredient; and
assign a higher weight to a feature corresponding to the active ingredient compared to weights corresponding to other features of the first AI engine.

13. The system of claim 9, wherein to generate the master formulation record, the at least one processor is further configured to:
predict one or more attributes of the master formulation record, wherein the attributes comprise at least one of an ingredient list, an expiration date, an equipment, a set of guidelines, and instructions to compound the preparation or drug product.

14. The system of claim 9, wherein the at least one processor is further configured to:
automatically correct one or more typographical errors in the recipe data before the comparing.

15. The system of claim 9, wherein the at least one processor is further configured to: receive, from a user device, data that identifies a patient;
retrieve the recipe data associated with the patient; and
display one or more master formulation records associated with the patient.

16. A non-transitory computer-readable device having instructions stored thereon that, when executed by at least one computing device, causes the at least one computing device to perform operations comprising:
receiving recipe data for a compounded preparation or drug product;
comparing, using a first artificial intelligence (AI) engine, the recipe data with stored recipe data to determine whether a master formulation record corresponding to the recipe data is available;
generating, using a second AI engine, the master formulation record for the compounded preparation or drug product based on a determination that the master formulation record corresponding to the recipe data is not available;
receiving an input comprising an approval or a correction for the generated formulation record;
retraining the second AI engine based on the input; and
generating, using the retrained second AI engine, a missing master formulation record for another recipe data.

17. The non-transitory computer-readable device of claim 16, the operations further comprising:
determining a match score between the recipe data and the stored recipe data;
displaying a list of one or more recipe for a user to select on a user device, wherein the displayed one or more recipe have a respective match score above a threshold;
receiving a selection from the user, wherein the selection indicates a correct match between the recipe data and the stored recipe data; and
retraining the first AI engine based on the selection of the user.

18. The non-transitory computer-readable device of claim 17, the operations further comprising:
determining that a first ingredient included in the recipe data and a second ingredient included in the stored recipe data are exchangeable based on the selection received from the user;
adding the first ingredient and the second ingredient to a synonyms database; and
retraining the first AI engine using the synonyms database.

19. The non-transitory computer-readable device of claim 16, the comparing comprising: parsing the recipe data to extract an active ingredient; and
assigning a higher weight to a feature corresponding to the active ingredient compared to weights corresponding to other features of the first AI engine.

20. The non-transitory computer-readable device of claim 16, the operations further comprising:
predicting one or more attributes of the master formulation record, wherein the attributes comprise at least one of an ingredient list, an expiration date, an equipment, a set of guidelines, and instructions to compound the compounded preparation or drug product.
